(19) United States
(12) Patent Application Publication
(10) Pub. No.: US 2004/0015153 A1
(43) Pub. Date: Jan. 22, 2004

(54) Fitted protective hip brief

(76) Inventor: Frederick T. Strobl, Wayzata, MN (US)

Correspondence Address: MERCHANT & GOULD PC, P.O. BOX 2903, MINNEAPOLIS, MN, 55402-0903, US

(21) Appl. No.: 10/302,603

(22) Filed: Nov. 21, 2002

Related U.S. Application Data
(63) Continuation of application No. 10/104,875, filed on Mar. 21, 2002, now abandoned.
Application No. 10/104,875 is a continuation of application No. 09/098,963, filed on Jun. 18, 1998, now abandoned.

(51) Int. Cl. A61M 1/00

(52) U.S. Cl. 604/540

(57) ABSTRACT

A brief for padding a wearer's hips to reduce the risk of hip fracture. The brief comprises a front panel having first and second side edges and a rear panel having first and second side edges. The first side edge of the rear panel is connected to the first side edge of the front panel, and the second side edge of the rear panel is connected to the second side edge of the front panel, thereby forming first and second opening for receiving the wearer's legs. Shock-absorbing padding lines at least part of the rear panel. The shock-absorbing padding extends from a position proximal the first edge of the rear panel to a position proximal the second side edge of the rear panel and at least to a position between the two leg openings.

### TECHNICAL FIELD

The present invention is directed to a device for protecting a person's hip, and more particularly, to a fitted protective hip brief.

### BACKGROUND

Every year in the United States, about 250,000 people suffer from hip fractures. 90% of those people are over the age of 50. As the population of our country ages, the number of people suffering hip fractures is projected to double by the year 2040. Of people who suffer from a hip fracture, only 50-65% regain their previous level of mobility. Additionally, a study of 2624 patients showed that only 24% of people suffering from a hip fracture returned home after recovering a nursing home. An even more tragic result of hip fractures is that 14-36% of elderly patients die within one year after sustaining hip fracture.

The majority of hip fractures are caused by trauma rather than weakening of the bone. When a person falls, or takes some other blow to the hip, kinetic energy from the impact is transmitted to the hip bone and causes a fracture. Various devices have been proposed to prevent hip fractures that result from such trauma, but none of these device have achieved any level of widespread acceptance. In fact, one study showed that as few as 24% of the people given hip protectors as part of the study had regularly worn the hip protectors. Possible reasons that so many people participating in the study failed to wear their hip protectors include poor difficulty of use, poor fit, and questionable efficacy.

In addition to the susceptibility of falling, many older people suffer from incontinence, which can be a very embarrassing condition, especially in public areas and social situations. Thus, many elderly people wear diapers or some type of absorbent lining to prevent any noticeable leaking through their clothing. In addition to the elderly, both incontinence and a tendency to fall is also common among younger people who suffer from conditions such as spinal cord injuries, multiple sclerosis, and other nervous system diseases.

Therefore, there is a need for a hip protection device that is easy to use. There is a further need for a hip protection device that is form fitting and thus more comfortable to wear. There is also a need for a hip protection device that a person can wear in a discrete manner. There is yet another need for a device that can both protect the wearer's hip and serve as a diaper.

### SUMMARY

In general terms the present invention is directed to a form-fitting, hip-protection device for absorbing kinetic energy and thus reducing the risk of fracturing a hip. One possible embodiment of the invention is directed toward a brief for padding a wearer's hips. The brief comprises a front portion, a rear portion, and a middle portion. The middle portion extends between the front and rear portions. The front portion is configured and arranged to be worn proximal to the wearer's abdomen. The rear portion configured and arranged to be worn proximal to the wearer's buttocks. The middle portion is configured to extend between the wearer's legs. A shock-absorbing padding lines the rear portion.

Another possible embodiment of the invention comprises a front panel having first and second side edges and a rear panel having first and second side edges. The first side edge of the rear panel is connected to the first side edge of the front panel, and the second side edge of the rear panel is connected to the second side edge of the front panel, thereby forming first and second openings for receiving the wearer's legs. Shock-absorbing padding lines at least part of the rear panel. The shock-absorbing padding extends from a position proximal the first edge of the rear panel to a position proximal the second side edge of the rear panel and at least to a position between the two leg openings.

### DESCRIPTION OF THE DRAWINGS

FIG. 1 is a perspective view of a brief incorporating the present invention as is worn by a person;

FIG. 2 is a top elevational view illustrating the inner surface of the brief shown in FIG. 1;

FIG. 3 is a top elevational view illustrating the inner surface of an alternative embodiment of the brief shown in FIGS. 1 and 2;

FIG. 4 is a top elevational view illustrating the inner surface of an alternative embodiment of the brief shown in FIGS. 1 and 2; and

FIG. 5 is a partial cross-sectional view taken along line 5-5 in FIG. 4.

### DETAILED DESCRIPTION

The present invention will be initially described in general terms. Various embodiments of the present invention, including the preferred embodiment, then will be described in detail with reference to the drawings wherein like reference numerals represent like parts and assemblies throughout the several views. Reference to the described embodiments is not meant to limit the scope of the invention, which is limited only by the scope of the appended claims.

In general terms, the present invention is directed to a form-fitted brief that includes padding formed from shock-absorbent material. This invention has several advantages. For example, it is comfortable to wear and is not intrusive. As a result, people who need protection are more likely to wear this hip protection device on a regular basis. Another advantage is that it has a low profile and can be worn under street cloths, much like underwear. Yet another advantage is that the brief securely holds the shock-absorbent material and prevents it from slipping out of position to expose the hip. Furthermore, alternative embodiments of the brief can integrate an absorbent layer so that the protective brief also functions as a diaper for those people who suffer from incontinence as well as have a susceptibility to falling and fracturing their hip.

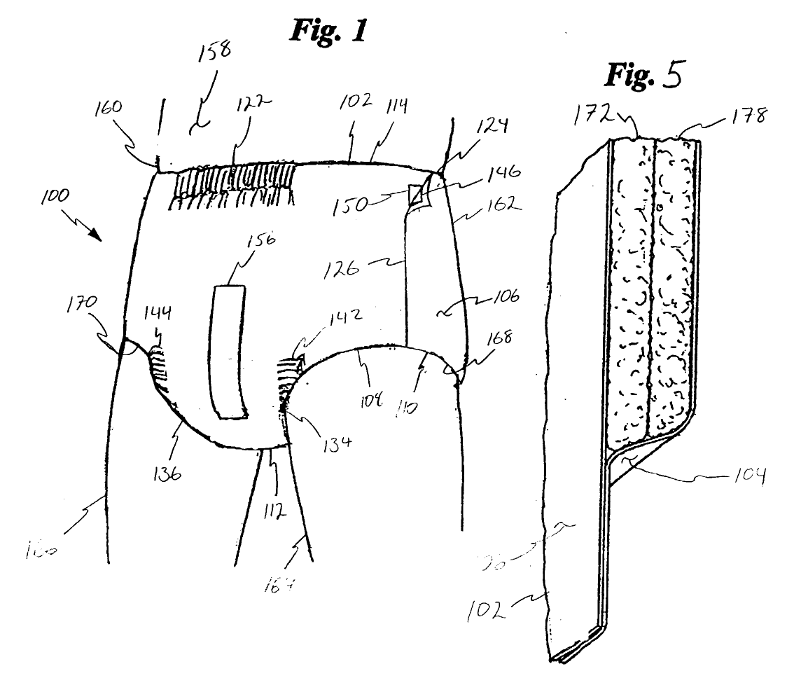

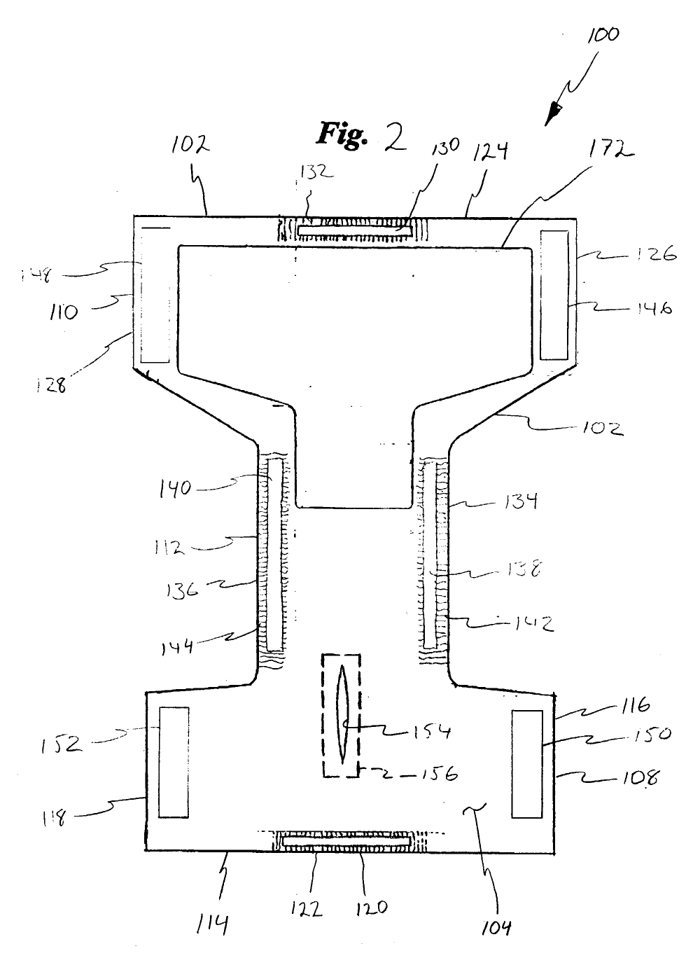

Referring now to FIGS. 1 and 2, a brief, generally shown as 100, has an outer shell 102 that includes an inner surface 104, an outer surface 106, a front portion 108, a rear portion 110, and a middle portion 112. The front portion 108 has a front edge 114, a first side edge 116, and a second side edge 118. The front portion 108 and a region of the middle portion 112 form a front panel, and the rear portion 110 and a region of the middle portion 112 for a rear panel. In this configuration, the front and rear panels are joined in the middle portion 112.

A front elastic band 120 or member is mounted on the front edge 114 of the front portion 108. Additionally, gathers 122 are formed in the front edge 114 along the length of the elastic band 120. The elastic band 120 and gathers 122 permit the front portion 108 to comfortably conform to the shape and size of the wearer's abdomen.

Similarly, the rear portion 110 has a rear edge 124, a first side edge 126, and a second side edge 128. A rear elastic band 130 or member is mounted on the rear edge 124 of the rear portion 110. Gathers 132 are formed in the rear edge 124 along the length of the elastic band 130. The elastic band 130 and gathers 132 permit the rear edge 124 to conform to the shape and size of the wearer's lower back.

The middle portion 112 is substantially narrower than the front and rear portions 108 and 110 and is sized to extend between a wearer's legs. The middle portion 112 has first and second side edges 134 and 136. A first elastic band 138 or member extends along the first side edge 134 of the middle portion 112, and a second elastic band 140 or member extends along the second side edge 136 of the middle portion 112. Gathers 142 and 144 are formed in the first and second side edges 134 and 136, respectively of the middle portion 112 along the first and second elastic bands 138 and 140, respectively. The gathers 142 and 144 permit the middle portion 112 to adjust to the size and contour of the wearer's legs and crotch.

One advantage of the gathers and elastic bands is that they permit the brief 100 to adjust to the size and shape of the wearer's body. If the brief 100 is also serving a diaper, the gathers and elastic bands help to retain moisture within the brief 100. Although gathers and elastic bands are shown along the front edge 114, the rear edge 124, and the side edges 134 and 136 of the middle portion 112, an alternative embodiment might include gathers or elastic members in only some of these locations or not include any gathers or elastic members at all.

Hook and loop fastener 146 such as a Velcro™ brand fastener is connected to the first and second side edges of the front and rear portions. As shown in FIG. 2, a first strip 140 of hook material is attached to the first side edge 126 of the rear portion 110 and a second strip 148 of hook material is attached to the second side edge 128 of the rear portion 110. A first strip 150 of loop material is attached to the first side edge 116 of the front portion 108 and a first strip 152 of loop material is attached to the second side edge 118 of the front portion 108.

An elongated opening 154 is defined in the front portion 108 and flap 156 is connected to the outer surface 106 of the front portion 108. The elongated opening 154 has a first end 158 oriented toward the front edge 114 of the front portion 108 and a second end 160 oriented toward the middle portion 112 of the brief 100. The flap 156 covers the elongated opening 154, and is connected to the front portion 108 along three of its four sides for access to the elongated opening 154. This opening 154 is advantageous for male wearer's so that they do not need to remove the brief 100 each time that they go to the bathroom. Other possible embodiments do not include such an elongated opening 154 and flap 156.

When worn as shown in FIG. 1, the front portion 108 lays against the wearer's abdomen 158 and the front edge 114 is positioned proximal the waist line 160. The rear portion 110 lays against the wearer's buttocks 162. The middle portion 112 extends between the wearer's legs 164 and 166. Additionally, the first strip of hook material 146 engages the first strip of loop material 150 and holds the first side edge 126 of the rear portion 110 against the first side edge 116 of the front portion 108. Similarly, the second strip of hook material 148 engages the second strip of loop material 152 and holds the second side edge 128 of the rear portion 110 against the second side edge 118 of the front portion 108. In this position, the brief 110 forms a first opening 168 through which the left leg 164 passes and a second opening 170 through which the right leg 166 passes.

Returning to FIG. 2, a layer of shock-absorbent material 172 lines the inner surface 104 of the rear portion 110. The layer of shock-absorbent material 172 extends from a position proximal the first side edge 126 to a position proximal the second side edge 128, and from the rear edge 124 to a position between the first and second side edges 134 and 136 of the middle portion 112. When a brief 100 having this configuration of shock-absorbent material 172 is worn as shown in FIG. 1, the shock-absorbent material 172 will extend from the side of the right hip to the side of the left hip, and down below the tail bone. The shock-absorbent material 172 will then protect the hips and tail bone from trauma if the wearer fall backwards, which is the most typical way to fall.

In an alternative embodiment, the layer of shock-absorbent material covers substantially the entire inner surface 104 of the shell 102. In this embodiment, the shock-absorbent layer extends from the front edge 114 to the rear edge 124, from the first side edge 126 of the rear portion 110 to the second side edge 128 of the rear portion 110, and from the first side edge 116 of the front portion 108 to the second side edge 118 of the front portion 108. An advantage of this embodiment is that it provides complete coverage and protection for the hips, pelvis, and tail bone. It also provides protection when the wearer falls forward.

The layer shock-absorbent material 172 can be formed from any material that will absorb energy that results from an impact such as a fall. One possible shock-absorbent material is foam. Another possible shock-absorbent material is one that includes a plurality of fluid filled chambers or passages. The fluid can be liquid or gas. Additionally, the chambers or passages can be of various sizes, shapes, and configurations. An example of shock-absorbent material is disclosed in U.S. Pat. No. 5,034,998, entitled Protective Device For Reducing Injury From Falls, the disclosure of which is hereby incorporated by reference.

In an alternative embodiment, the first and second side edges 126 and 128 of the rear portion 112 are integrally formed with the first and second side edges 116 and 118 of the front portion 108, thereby by forming the first and second openings 168 and 170 through which the legs 164 and 166 can pass. The front and rear edges 114 and 124 form a waste band. In this alternative embodiment, the briefs 100 are pull-ups, similar to traditional underwear. Additionally, in this alternative embodiment, the briefs 100 can define a score, perforations, or an indented line extending from the waste band to the leg opening. The briefs can then be torn along the score, perforation, or indented line for easy removal.

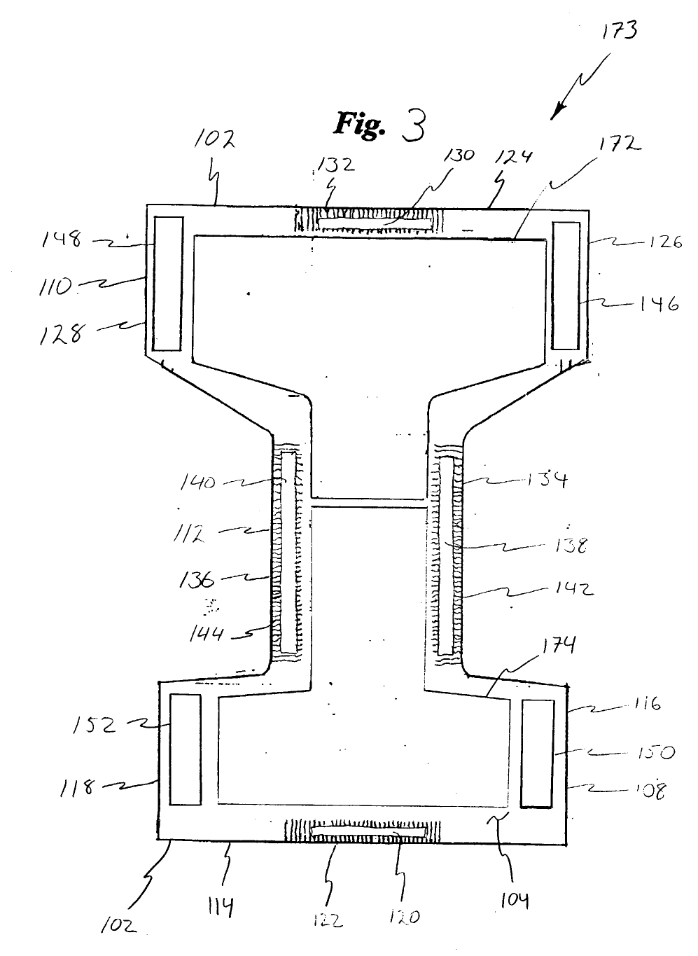

Referring now to FIG. 3, an alternative embodiment, generally shown as 173 is substantially similar to the embodiment shown in FIG. 2 and includes an outer shell 102 having an inner surface 104, a front portion 108, rear portion 110, and middle portion 112; a layer of shock-absorbent material; and first and second hook and loop fasteners 146, 148, 150, and 152. The shell 102 is made from a liquid-impervious material. In an alternative embodiment, the shell is impervious to substantially all fluids, including gases and liquids. Additionally, a liquid-absorbent layer 174 lines the front portion 108 and extends from the first side edge 116 to the second side edge 118, and from the front edge 114 to a position between the side edges 134 and 136 of the middle portion 112 and adjacent to the layer of shock-absorbent material 172. The liquid-absorbent material 174 can be formed from material similar to that used for diapers, and can have alternative configurations and shapes. An advantage of this embodiment is that the brief 173 functions both as a hip protector and a diaper. Thus, people who suffer from incontinence and have a susceptibility to falling do not have to wear multiple layers of protective clothing.

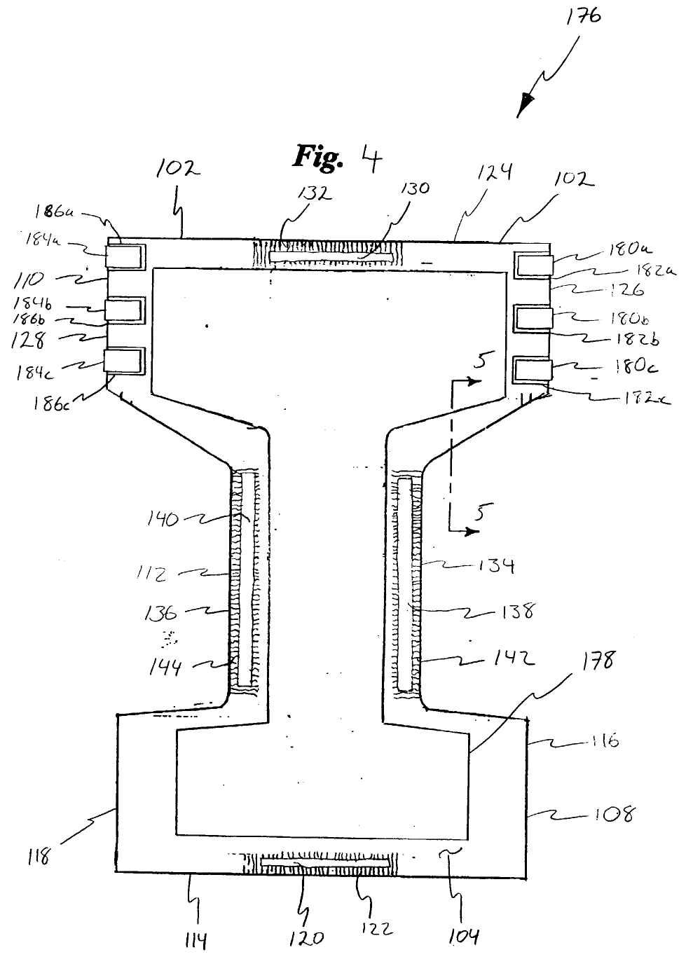

Referring now to FIGS. 4 and 5, yet another alternative embodiment, generally shown as 176, is substantially similar to the embodiment shown in FIG. 2 and includes an outer shell 102 having an inner surface 104, a front portion 108, rear portion 110, and middle portion 112; a layer of shock-absorbent material 172; and first and second hook and loop fasteners 146, 148, 150, and 152. The shell 102 is made from a liquid-impervious material. Additionally, a liquid-absorbent layer 178 lines substantially the entire inner surface 104 of the shell 102 and extends from the front edge 114 to the rear edge 124, from the first side edge 126 of the rear portion 110 to the second side edge 118 of the rear portion 110, and from the first side edge 116 of the front portion 108 to the second side edge 118 of the front portion 108. In this embodiment, the layer of shock-absorbent material 172 is positioned between the liquid-absorbent layer 178 and the shell 102.

Additionally, the embodiment shown in FIG. 4 has an alternative fastener formed of adhesive strips. There are three adhesive strips 180a-188c mounted along the first side edge 126 of the rear portion 110. The adhesive strips 180a-180c are affixed to the outer surface 106 and extend around the side edge 126 where they are affixed to a releasable surface 182a-182c. In use, the first side edge 126 of the rear portion 110 placed in an overlapping position over the first side edge 116 of the front portion 108, the adhesive strips 180a-180c are then peeled off the releasable surface 182a-182c and affixed to the outer surface 106 of the shell 102 along the front portion 108. There are three adhesive strips 184a-184c and releasable surfaces 186a-186c similarly mounted to the second side edge 128 of the rear portion 110. Yet other possible embodiments may have other types of fasteners.

Additionally, the outer shell 102 can be made from a variety of different materials depending on the embodiment of the invention. For example, in the embodiment shown in FIG. 2, the shell 102 can be formed from a material that breaths such as cotton In the embodiments shown in FIGS. 3-5 in which an absorbent material is included so that the brief functions as diaper as well as a hip protector, the outer shell 102 is formed from a liquid imperious material such as a polymer.

Although the description of the various embodiments and methods have been quite specific, it is contemplated that modifications could be made without deviating from the spirit of the present invention. Accordingly, it is intended that the scope of the present invention be dictated by the appended claims, rather than by the description of the various embodiments and methods.

The claimed invention is:

1. A brief for padding a wearer's hips to reduce the risk of hip fracture, the brief comprising:
a front portion, a rear portion, and a middle portion extending between the front and rear portions, the front portion configured and arranged to be worn proximal to the wearer's abdomen, the rear portion configured and arranged to be worn proximal to the wearer's buttocks, and the middle portion is configured to extend between the wearer's legs; and
a shock-absorbing padding lining the rear portion.

2. The brief of claim 1 wherein the wearer has a hip joint and the hip joint has a rear portion, further wherein the rear portion has first and second side edges, the padding extending between a position proximal the first side edge and a position proximal the second side edge, the padding thereby being configured to cover the rear portion of the wearer's hip joint.

3. The brief of claim 2 wherein the wearer has a tail bone, the padding further extending over at least part of the middle portion of the brief, the padding thereby being configured to cover the wearer's tail bone.

4. The brief of claim 2 wherein the brief has an inner surface, the padding being configured and arranged to cover substantially the entire inner surface of the brief.

5. The brief of claim 1 wherein the front, middle, and rear portions are formed from a liquid-impervious sheet and the shock-absorbing padding is mounted to the sheet, the brief further comprising a layer of absorbent material, the absorbent material lining the front portion, the brief thereby forming a diaper.

6. The brief of claim 5 wherein brief has an inner surface and the layer of absorbent material covers substantially the entire inner surface, the shock-absorbing padding being positioned between the sheet and the absorbent material.

7. The brief of claim 6 wherein the middle portion has first and second side edges, the brief further comprising a first and second elastic bands, the first elastic member being connected to the middle portion proximal to the first edge and the second elastic member being connected to the middle portion proximal to the second edge, wherein the first and second edges of the middle portion are configured to conform to the wearer's crotch.

8. The brief of claim 7 wherein the sheet forming the middle portion has gathers extending along the first and second elastic members wherein the first and second side edges of the middle portion are expandable.

9. The brief of claim 6 wherein the front portion has a front edge and the rear portion has a read edge, the front and rear edges forming a waistband, the brief further comprising an elastic member extending along at least a part of the front edge and gathers along the front edge, the front edge thereby being configured to conform to the wearer's abdomen.

10. The brief of claim 6 wherein the brief further comprises an elastic member extending along at least a part of the rear edge and gathers along the rear edge, the rear edge thereby being configured to conform to the wearer's lower back.

11. The brief of claim 6 wherein the front portion defines an opening.

12. The brief of claim 11 wherein the front portion has a front edge, the rear portion has a rear edge, and the opening is elongated thereby forming oppositely disposed ends, one end of the opening being oriented toward the front edge and the oppositely disposed end being oriented toward the rear edge, the brief further comprising a flap connected to the front portion, the flap covering the opening.

13. The brief of claim 1 wherein the front portion has first and second side edges and the rear portion has first and second side edges, the brief further comprising:
at least one hook and loop fastener releasably connected between the first edge of the front portion and the rear edge of the rear portion; and
at least one hook and loop fastener releasably connected between the second edge of the front portion and the second edge of the rear portion.

14. The brief of claim 1 wherein the front portion has first and second side edges and the rear portion has first and second side edges, the brief further comprising:
at least one adhesive strip configured and arranged to extend between the first edge of the front portion and the rear edge of the rear portion; and
at least one adhesive strip configured and arranged to extend between the second edge of the front portion and the second edge of the rear portion.

15. The brief of claim 1 wherein the front portion has first and second side edges and the rear portion has first and second side edges, the first edge of the front portion being connected to the first edge of the rear portion, and the second edge of the front portion being connected to the second edge of the rear portion, thereby forming a pull-up brief.

16. The brief of claim 15 wherein the brief defines a first score between the first edge of the front portion and the first edge of the rear portion and a second score between the second edge of the front portion and the second edge of the rear portion.

17. A brief for padding a wearer's hips to reduce the risk of hip fracture, the brief comprising:
a front panel having first and second side edges;
a rear panel having first and second side edges, the first side edge of the rear panel being connected to the first side edge of the front panel, and the second side edge of the rear panel being connected to the second side edge of the front panel, thereby forming first and second opening for receiving the wearer's legs;
shock-absorbing padding lining at least part of the rear panel, the shock-absorbing padding extending from a position proximal the first edge of the rear panel to a position proximal the second side edge of the rear panel and at least to a position between the two leg openings.

18. The brief of claim 17 wherein the front and rear panels form an inner surface and the shock-absorbing padding lines substantially the entire inner surface.

19. The brief of claim 18 wherein the front panel defines an elongated opening and the brief further comprises a flap, the flap being connected to the front panel and positioned over the elongated opening.

20. The brief of claim 18 wherein the front and rear panels are formed from material that is impervious to at least liquids, the brief further comprising a layer of absorbent material lining at least a portion of the front panel, the layer of shock-absorbent material being positioned between the front panel and the absorbent material.
